(19) United States
(12) Patent Application Publication
(10) Pub. No.: US 2015/0191689 A1
(43) Pub. Date: Jul. 9, 2015

(54) USE OF PURE OXYGEN IN AEROBIC FERMENTATION FOR OPTIMUM PERFORMANCE

(71) Applicant: Sudhir R. Brahmbhatt, Glencoe, MO (US)

(72) Inventor: Sudhir R. Brahmbhatt, Glencoe, MO (US)

(21) Appl. No.: 14/490,002

(22) Filed: Sep. 18, 2014

Related U.S. Application Data
(60) Provisional application No. 61/924,473, filed on Jan. 7, 2014.

(51) Int. Cl. C12M 1/00; C12M 1/34; C12M 1/02

(52) U.S. Cl. CPC C12M 29/06; C12M 41/22; C12M 41/00; C12M 29/08

(57) ABSTRACT

A method of economically performing a fermentation process in an air lifted or mechanical fermenter with significant increase in productivity and yield. During a lag phase of the process, air is used in a fermenter (10) as organisms in a media (F) adjust to the environment in the fermenter. During a subsequent organism growth phase, only pure oxygen (O₂) is supplied to the fermenter so as to provide the maximum amount of oxygen required by the organisms. During a stationary phase, an oxygen enriched environment is provided and this minimizes pure oxygen use. During a final phase of the process, regular air, but not any pure oxygen, is introduced into the fermenter. Appropriate controls are used to control both the supply of air, pure oxygen, and a combination of the two. The result is a cost effective, optimum and efficient with significant increase in productivity and yield in fermentation process.

### CROSS REFERENCE TO RELATED APPLICATIONS

This application is based on and claims the benefit of provisional patent application 61/924,473 filed Jan. 7, 2014.

### STATEMENT REGARDING FEDERALLY SPONSORED RESEARCH

N/A

### BACKGROUND OF THE INVENTION

This invention is directed to optimizing the economics and performance of aerobic fermentation systems; and, in particular, depending on the stage of a fermentation cycle in such a system, the injection of pure oxygen into the fermenter for optimum performance and efficient use of the oxygen.

A substantial amount of work has been done in the fermentation industry to increase the efficiency and productivity of a typical aerobic fermenter. Previous efforts in this regard include use of pure oxygen as the sole oxygen source in an aerobic fermenter, or in enriching an airline by which air is supplied to a fermenter with oxygen. But, oxygen enrichment has proven to be expensive due to the large volume of oxygen required and its limited impact; while, pure oxygen has demonstrated interesting benefits in the areas of productivity and yield.

It has been found, however, that the costs associated with using pure oxygen may also be cost prohibitive, especially if retrofitting a fermenter to use pure oxygen is necessary. The present invention is directed to further improving the economics associated with implementing a pure oxygen technology in a fermentation process. Since employing pure oxygen, as a single source of oxygen, requires modifying an existing aerobic fermenter, the innovative approach described herein helps reduce the cost for implementing a cost effective system which benefits from the use of pure oxygen.

### SUMMARY OF THE INVENTION

The present invention is directed to a method for more economically performing a fermentation process. A conventional fermenter may require minor modifications to facilitate the introduction of pure oxygen into the fermenter media.

At the beginning of a fermentation cycle; i.e., during the first or lag phase of the cycle, air is supplied to the fermenter as organisms adjust to the new environment when they are transferred from a seed fermenter to the production fermenter. During a second or growth phase of the cycle, only pure oxygen (O₂) is supplied to the fermenter so as to provide the maximum amount of dissolved oxygen (DO) required by organisms for growth in the media. In a third or stationary phase, an oxygen enriched air is injected into the fermenter media. This is because the demand for oxygen is reduced because the organisms are no longer multiplying, but basically producing the product. And during a final phase of the fermentation cycle, known as the death phase, regular air is injected into the media because the organisms are at the end of their life and their need for oxygen is minimal. Appropriate controls are used to control both the supply of air, pure oxygen, and also a combination of the two. The result is a cost effective, yet efficient, fermentation process. Those skilled in the art will understand that the technology described herein is not limited to mechanical fermenters, but also to air lifted or similar aerobic fermenters where air is used to supply oxygen for DO in the media.

Other objects and features will be in part apparent and in part pointed out hereinafter.

### BRIEF DESCRIPTION OF THE DRAWING

FIG. 1 is a simplified representation of a fermenter configured in accordance with the present invention; and,

FIG. 2 is a plan view of an exemplary oxygen injector inlet.

Corresponding reference characters represent corresponding parts in the views of the drawings.

### DETAILED DESCRIPTION OF INVENTION

The following detailed description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

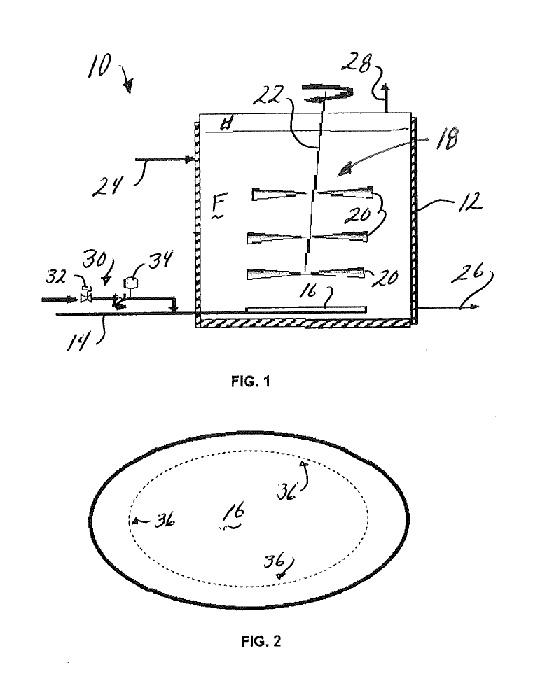

Referring to FIG. 1, in a typical mechanical aerobic fermenter generally indicated 10 comprises a closed jacketed vessel 12. As noted previously, the technology herein described also applies to air lifted fermenters. A key distinction between the two types of fermenters is the use of an agitation system in mechanical fermenter 10. An airline 14 which is made of an oxygen compatible material is used to inject air and oxygen, or oxygen enriched air, into the bottom of the vessel through an appropriately sized and oxygen compatible injector, or sparger or diffuser inlet, 16. An agitator assembly 18 includes a series of impellers 20 mounted along a shaft 22. A control mechanism (not shown) rotates shaft 22 causing the impellers to turn and agitate a media F within the vessel 12. Cooling media such as water is supplied through an inlet line 24 in the jacket of vessel 12 to maintain the fermenter media temperature. The cooling media is discharged from the vessel jacket through an outlet line 26. An exhaust of the fermenter is vented from the vessel through an outlet 28.

An aerobic fermentation cycle, as previously noted, involves four phases: a lag phase, a growth phase, a stationary phase, and a “death” or final phase. In accordance with the invention, one executing such a cycle can continue to use existing airline 14 which is now adapted for flowing air, pure oxygen (O₂), or a combination of the two into the container. If airline 14 is compatible for use with pure oxygen, then the operator can connect a pure oxygen line, indicated generally 30 in FIG. 1, to airline 14. In this regard, line 30 includes appropriate valving 32 and flow controls 34 for appropriate enrichment level and routing the pure oxygen into airline 14 for its subsequent discharge into container 12 through sparger or injector 16.

Further in accordance with the invention, sparger or diffuser 16 needs to be appropriate for generating small bubbles. This is for effective dissolution of oxygen into the media. A ceramic diffuser may, for example, provide the desired bubble size. As shown in FIG. 2, one such configuration of a sparger 16 having holes sufficiently small in size is oval shaped when viewed in plan. A number of spaced openings 36 are formed on the circumference of the sparger. The openings are arranged in an oval pattern and the size of the openings are such that the oxygen bubbles emanating from the sparger through these openings are minute in size and are directed in all directions.

Operation of a fermentation system employing fermenter 10 is as follows:

First, during the initial, lag phase of the process, valve 32 in oxygen line 30 is closed, so that only air flowing through airline 14 is introduced into container 12 via sparger 16. At this time, organisms in the media F within container 12 adjust to the environment within the container (temperature, pressure, composition, pH etc.).

Second, during the growth phase of the process, air flow through airline 14 is stopped and valve 32 is opened, and only pure oxygen now flows into the container via sparger 16. At this time, the organisms within the media are growing and multiplying at a desired rate and producing product in accordance with predetermined growth factors. The demand for the dissolved oxygen (DO) is significant in this phase, and pure oxygen provides sufficient dissolved oxygen to ensure no limitation of oxygen in increasing the population of organisms. In the absence of nitrogen or air, pure oxygen, as a single source for oxygen, has more than five time dissolving ability into the media, this allowing an operator to provide sufficient DO without any difficulty.

Third, during the stationary phase of the process, air again flows through airline 14 into the container, while the amount of pure oxygen flowing through line 30 is controlled so that oxygen enriched air, but not pure oxygen, flows into the container. This approach allows the operator to reduce their use of pure oxygen.

Finally, in the final phase of the process, pure oxygen flow is terminated and only air is introduced into container 12. At the end of this phase, the fermentation process is complete.

Those skilled in the art will appreciate that implementation of the invention will minimize any necessary modifications to an existing fermenter so as to be cost effective. Accordingly, the capabilities and capacities of various components must be determined prior to converting to use of pure oxygen in the system.

First, existing airlines 14 need to be compatible for use with pure oxygen. In this regard, for many pharmaceutical and biotechnical processes, the airlines 14 should be of stainless steel. If an airline 14 is compatible, pure oxygen line 30 is connected to it downstream of an air compressor (not shown). Next, the sparger 16 used should be one that produces oxygen bubbles as small, i.e., minute, as possible. For this purpose, a ceramic diffuser or similar sparger 16 is preferably used. Because of the size of the holes 36 required to achieve this affect, proper installation and maintenance/repair procedures need to be followed to avoid their blockage. This may require the use of an alternate air supply when sparger or injector or diffuser 16 is not used. This will maintain a positive pressure in line 14 and avoid particles from blocking the sparger holes or diffuser surface.

Once the above has been accomplished so that fermenter 10 is configured as shown in the drawings, operation is as follows:

Lag phase: Air is used in the fermenter as organisms adjust to the environment in the fermenter.

Growth phase: Only pure oxygen is used during this phase to provide the maximum amount of oxygen required by the organism.

Stationary phase: An oxygen enrichment environment is now provided to minimize oxygen use.

Death phase: Air is used for this phase.

To ensure controlled use of pure oxygen and avoid any unsafe operating conditions in fermenter 10, the pure oxygen input is controlled such that the level of pure oxygen in a head space H of the fermenter is well below allowable, safe limits. This is because of the potential presence of ammonia in the head space due to a nitrogen compound being used as a nutrient in the fermentation process. Likewise, the level of Dissolved Oxygen (DO) in the fermenter media is maintained such as to avoid excessive DO levels. This is because sometimes high DO levels may affect the performance of organisms. Also, pH and CO2 levels in the media are monitored and maintained such as to avoid any unfavorable conditions for the organisms present in the media. In mechanical aerobic fermentation processes, the need for agitation used to increase the mixing capability will be reduced with higher levels of DO. This is because the pure oxygen will easily diffuse into low DO areas, even with less agitation. This should result in energy savings. Under low agitation conditions, CO₂ has been found to release from media F without causing any detrimental effect on organisms present in the media.

When pure oxygen is used, exhaust flow through outlet 28 is significantly reduced because of the absence of air. In this situation, padding head space H with air is recommended. This permits the operator a) to dilute the oxygen level in case of any breakthrough of oxygen through media F or excessive use of pure oxygen; and b) to have enough fermenter vent flow to analyze and monitor the head space composition.

The advantage of using pure oxygen as described above has been found to result in as much as a 30% or more increase in productivity; i.e., the final mass of product per unit of volume per EFT (Effective Fermentation Time) and 10%+yield improvements i.e. total mass of product per total substrate consumed.

In view of the above, it will be seen that the several objectives and advantages of the present disclosure have been achieved and other advantageous results have been obtained.

What I claim is:

1. A multi-phase fermentation process comprising:
a lag phase during which air is supplied to organisms present in a media within a fermenter to assist the organisms in adjusting to an environment in a fermenter;
a growth phase during which only pure oxygen (O₂) is supplied to the fermenter so to provide a maximum amount of oxygen required by the organisms present in the media to facilitate their growth;
a stationary phase during which both air and a reduced amount of pure oxygen are mixed together and introduced into the fermenter so as to provide an oxygen enriched environment while minimizing the use of pure oxygen; and,
a final phase of the process in which regular air, but not any pure oxygen, is introduced into the fermenter so to provide a cost effective, yet efficient, fermentation process.

2. The process of claim 1 in which the fermenter includes an airline for supplying air to the fermenter and a line for supplying pure oxygen to the fermenter is connected to the airline for supplying pure oxygen to the fermenter through the airline.

3. The process of claim 2 further including valving and flow controls in the pure oxygen supply line to control the flow of pure oxygen to the airline.

4. The process of claim 3 further including controls to control both the supply of air, pure oxygen, and a combination of the two to the fermenter during the respective phases of the fermentation process.

5. The process of claim 1 which is implemented in either a mechanical or air lifted fermentation system.

6. A fermenter for use in a fermentation process comprising:
a closed container in which a media is contained;
an airline for injecting air, oxygen, and air/oxygen into the container through one of a diffuser, sparger, or injector located within the container; and,
a line for supplying pure oxygen to the fermenter, said pure oxygen supply line being connected to the airline for supplying pure oxygen to the fermenter through the airline.

7. The fermenter of claim 6 further including means for supplying cooling water to and removing the cooling water from a jacket of the container, and an exhaust outlet for venting from the container.

8. The fermenter of claim 6 for performing a multi-phase fermentation process having a lag phase during which air is supplied to organisms present in a media within a fermenter to assist the organisms in adjusting to an environment in a fermenter; a growth phase during which only pure oxygen (O₂) is supplied to the fermenter so as to provide a maximum amount of dissolved oxygen required by the organisms present in the media to facilitate their growth; a stationary phase during which both air and a reduced amount of pure oxygen are introduced into the fermenter so to provide an oxygen enriched environment while minimizing the use of pure oxygen; and, a final phase in which regular air, but no pure oxygen, is introduced into the fermenter, the fermenter providing a cost effective, yet efficient with high productivity and yield in a fermentation process depending on the organisms.

9. The fermenter of claim 6 further includes valving and flow controls in the pure oxygen supply line to control the flow of pure oxygen to the airline.

10. The fermenter of claim 9 further including controls to control both the supply of air, pure oxygen, and a combination of the two to the fermenter during the respective phases of the fermentation process

11. The fermenter of claim 10 further including controls to monitor a head space within the container and exhaust oxygen levels.

12. The fermenter of claim 6 which is either a mechanical or air lifted fermenter.

13. The fermenter of claim 12 further including an agitator assembly for agitating the media within the container if the fermenter is a mechanical fermenter.
